(19) United States
(12) Patent Application Publication
(10) Pub. No.: US 2019/0214122 A1
(43) Pub. Date: Jul. 11, 2019

(54) KNOWLEDGE DISCOVERY FROM SOCIAL MEDIA AND BIOMEDICAL LITERATURE FOR ADVERSE DRUG EVENTS

(71) Applicant: KONINKLIJKE PHILIPS N.V., EINDHOVEN (NL)

(72) Inventors: Kathy Mi Young Lee, Westford, MA (US); Oladijemi Feyisetan Farri, Yorktown Heights, NY (US); Sheikh Sadid Al Hasan, Cambridge, MA (US); Vivek Varma Datla, Ashland, MA (US); Junyi Liu, Windham, NH (US)

(21) Appl. No.: 16/325,646

(22) PCT Filed: Aug. 17, 2017
(86) PCT No.: PCT/EP2017/070814

§ 371 Date: Feb. 14, 2019

Related U.S. Application Data
(60) Provisional application No. 62/377,778, filed on Aug. 22, 2016.

(51) Int. Cl. G16H 20/10; G16H 15/00

(52) U.S. Cl. CPC G16H 20/10; G16H 15/00

(57) ABSTRACT

In adverse drug event (ADE) monitoring and reporting, drug-related messages (60) are detected in one or more social media message streams as messages that include a name of a monitored drug. ADE reports (62) are extracted from the drug-related messages using an ADE classifier (46). The extracted ADE reports are validated by comparison with known ADEs of the monitored drug stored in an ADE knowledge base (64). Extracted ADE reports that fail the validating are collected in a non-validated ADE reports database (72). A report (74) is generated including information on at least one previously unrecognized ADE for which extracted ADE reports in the non-validated ADE reports database satisfy a previously unrecognized ADE criterion (in terms of number of messages or number of unique patients reporting the ADE).

### FIELD

The following relates generally to the pharmaceutical arts, pharmaceutical testing arts, pharmacovigilance arts, and related arts.

### BACKGROUND

In the United States, the approval process for a new pharmaceutical includes assessment of efficacy of the drug for its intended use, as well as assessment of side effects (more generally “Adverse Drug Events” or ADE). These assessments are done by way of controlled clinical trials. These studies employ relatively small test populations, which can limit the ability to uncover all ADEs during the clinical trials. To address this issue, pharmaceutical and regulatory organizations employ post-market surveillance programs to capture previously undiscovered side effects by monitoring use of the drug in the larger population of patients.

However, post-market ADE surveillance systems suffer from under-reporting and significant time delays in data processing, resulting in high incidence of unidentified adverse events related to medication use. Under-reporting is a consequence of reliance primarily upon self-reporting by patients, doctors, or medical institutions. This self-reporting is a secondary task for these individuals and institutions, whose primary concern is the welfare of the patient. It is commonplace for doctors to be so busy with the welfare of the patient (and other patients) that they forget to self-report. Many institutions do not have a consistent or established procedure for self-reporting. The self-reporting is typically provided without compensation or any expectation of compensation, and therefore, the patient, doctor, or institution is not strongly motivated to self-report.

Similar approaches for pharmacovigilance are also typically employed in countries other than the United States.

### SUMMARY

In one disclosed aspect, an adverse drug event (ADE) monitoring and reporting device comprises a computer programmed to perform an ADE monitoring and reporting method including: detecting drug-related messages in one or more social media message streams as messages that include a name of a monitored drug; extracting ADE reports from the drug-related messages using an ADE classifier; validating the extracted ADE reports by comparison with known ADEs of the monitored drug stored in an ADE knowledge base; collecting extracted ADE reports that fail the validating in a non-validated ADE reports database; and generating a report including information on at least one previously unrecognized ADE for which extracted ADE reports in the non-validated ADE reports database satisfy a previously unrecognized ADE criterion.

In another disclosed aspect, a non-transitory storage medium stores instructions readable and executable by a computer to perform an ADE monitoring and reporting method for a monitored drug having a set of known ADEs. The method comprises: identifying drug-related messages in one or more social media message streams wherein each drug-related message includes a name of the monitored drug; extracting ADE reports from the drug-related messages by classification of the drug-related messages using n-grams extracted from the drug-related messages as features of an ADE classifier; and identifying a previously unrecognized ADE that is not in the set of known ADEs for the monitored drug in response to an accumulation of extracted ADE reports indicating the previously unrecognized ADE.

In another disclosed aspect, an ADE monitoring and reporting method is performed for a monitored drug. The method comprises: identifying drug-related messages that include a name of the monitored drug; extracting ADE reports from the identified ADE reporting messages by classifying text of the drug-related messages using an ADE classifier; and outputting a report on the extracted ADE reports.

One advantage resides in providing for improved discovery of previously unrecognized adverse drug events (ADEs).

Another advantage resides in providing rapid discovery of previously unrecognized ADEs.

Another advantage resides in providing information on relative occurrence frequencies of various ADEs related to a drug.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically shows an illustrative pharmacovigilance device providing adverse drug event (ADE) monitoring and reporting.

FIGS. 2 and 3 diagrammatically show forward and backward propagation, respectively, through a convolutional neural network (CNN) employed by the pharmacovigilance device of FIG. 1.

FIG. 4 diagrammatically shows an ADE monitoring and reporting method suitably performed by the device of FIG. 1.

### DETAILED DESCRIPTION

Social media message streams such as Twitter and Facebook are used by many people worldwide to communicate about events in their daily lives. In the course of social media discourse, a user may send a message complaining about or otherwise discussing an adverse drug event (ADE) the social media user has experienced. Indeed, patients may be likely to send out social media messages about an ADE, since they use these services on a daily basis; by contrast, many patients are unaware of the reporting options available for filing “official” ADE reports, and may not take the time and effort to make such an official report even if they are aware of the reporting options.

In ADE monitoring and reporting approaches disclosed herein, real-time social media messages are monitored to detect ADE reporting messages, e.g. which specifically mention a monitored drug. The detected ADE reporting messages are validated by comparison with a knowledge base of known ADEs associated with the monitored drug. ADE reporting messages that cannot be so validated (because the reported ADE is not known to be associated with the monitored drug according to the knowledge base) are collected, and if enough such reports are accumulated this is reported as a previously unrecognized ADE. In some illustrative embodiments, natural language processing (NLP) and deep learning (DL) algorithms are used to detect ADEs in social media messages.

The knowledge base used for validating ADE reports extracted from social media messages may be generated from online medical knowledge sources such as PubMed articles, Pharmacology Text and Drug Formularies, Food and Drug Administration (FDA) adverse event databases, and drug side-effects information from publicly accessible sources such as WebMD or healthline. The approach can lead to the rapid discovery of previously unrecognized ADEs for the monitored drug that may have gone undetected in clinical trials and by other types of post-market surveillance.

As used herein, a “patient” is a person receiving (or registered to receive) medical care including taking and/or being prescribed the monitored drug. The term “patient” as used herein is not otherwise limited, for example is not limited to hospital patients, in-patients, patients diagnosed with any particular disease, patients under a particular doctor's care, nor is a “patient” limited to patients taking a prescription drug (i.e., the monitored drug may be a non-prescription or “over the counter” drug).

A “drug” as used herein indicates a medicine or other substance having, or intended to have, some desired physiological effect when ingested or otherwise administered to the patient. The desired “physiological effect” may, for example, be reduction of pain, treatment of an infection or disease, reducing swelling, inducing sleep, or so forth. The desired “physiological effect” may in some instances include a psychological effect, i.e. the drug may be a psychoactive drug. The desired physiological effect may in some instances be unpleasant for the patient, e.g. inducing vomiting for a clinically beneficial purpose and is not an ADE if the purpose of the drug is to induce the unpleasant effect.

The term “Adverse Drug Event” or ADE as used herein encompasses any effect of the drug that is other than the desired physiological effect and which may be in some way harmful to the patient and/or unpleasant or undesirable for the patient. ADEs may include, by way of non-limiting illustrative example: pain, discomfort, or the like; respiratory difficulty; cardiac arrhythmia; psychological effects such as hallucinations, depression, suicidal tendencies, or so forth; lifestyle impacts such as increased frequency of urination, loose bowels, or sleeping difficulty; morbidity effects such as increased likelihood of a heart attack, cancer, or other disease; adverse drug interactions, i.e. any of the foregoing correlated with taking both the monitored drug and a specific second drug; and so forth.

The term “previously unrecognized ADE” as used herein is in the context of the monitored drug—that is, the ADE is previously unrecognized as a potential adverse effect of the monitored drug, although it may be a known ADE for some other drug or drugs. Moreover, in the context of the ADE monitoring and reporting devices disclosed herein, a “previously unrecognized ADE” is more particularly an ADE which is not included in the set of known ADEs for the monitored drug which are stored in the ADE knowledge base leveraged by the ADE monitoring and reporting device. Thus, the “previously unrecognized ADE” might in fact have been recognized as associated with the monitored drug by some person(s), e.g. by some physician who is not in communication with the pharmaceutical company operating the ADE monitoring and reporting device—but the “previously unrecognized ADE” is not one of the known ADEs that are known to the ADE monitoring and reporting device.

A “social media message stream” as used herein is an Internet-based service that enables users to create and share content and thereby interact with each other. Users are typically assigned user accounts which are identified by a username (which may be fictitious or not personally identifying), and user accounts may be password-protected or otherwise secured. A social media message stream is generally public, although access may be limited in various ways, e.g. to individuals or entities having user accounts with the social network, or individual users may limit access to contacts of the user. A social media message stream may be general-purpose or may be domain-specific, e.g. forums dedicated to specific hobbies, interests, professions, medical conditions, or so forth. A “message” of a social media message stream is a unit of information generated by a user. Such a message is generally text-based, although it may also include multimedia content such as embedded images or videos, hyperlinks, audio files, or so forth. It is assumed here that the ADE monitoring and reporting device has at least read access to each social media message stream on which drug-related messages are detected.

In one embodiment, a data collection and preparation engine collects real-time social media (e.g. Twitter, Facebook) messages and filters ADE-related posts (with mentions on drug names and side effects) by referencing databases of drug names and side effects derived from the Unified Medical Language System (UMLS) Metathesaurus, and/or other medical/pharmacological dictionaries. The drug side effects database is optionally expanded by leveraging medical lay terminologies and building neural embeddings or the like to identify additional phrases related to side effects. Expert-annotated social media messages are generated indicating ADEs to be used as training data in the semi-supervised classification phase. A semi-supervised deep neural network architecture includes an unsupervised feature learning module trained on unlabeled social media data and medical concepts text to learn text features that is predictive of ADEs. The text features learned are used as features in a semi-supervised deep neural network to predict the labels (ADE or non-ADE) of new social media messages (test data). A knowledge-based validation engine builds an ADE knowledge base by combining online knowledge sources such as PubMed, WebMD and FDA databases for known ADE drug and side effect pairs. Social media messages identified as describing ADEs by the semi-supervised deep learning classifier are validated against the ADE knowledge base. If the ADE retrieved from the social media message correlates with the semantic properties of existing evidence in the knowledge base, the message is used to tune parameters of the ADE classifier. Otherwise, the non-validated ADE and corresponding social media message are stored in a knowledge repository while parsing other incoming messages for additional reports on the same ADE. If a non-validated ADE is reported by multiple social media messages (excluding re-distribution e.g. retweets) and exceeds an empirical reporting threshold, the system generates an alert/report on the newly found (i.e. previously unrecognized) ADE. In an alternative embodiment, the criterion for reporting a previously unrecognized ADE is based on the number of different patients reporting the ADE in social messages, rather than the total number of messages. This alternative approach can avoid the situation where a single patient who is very active on social media makes numerous posts reporting the same ADE event.

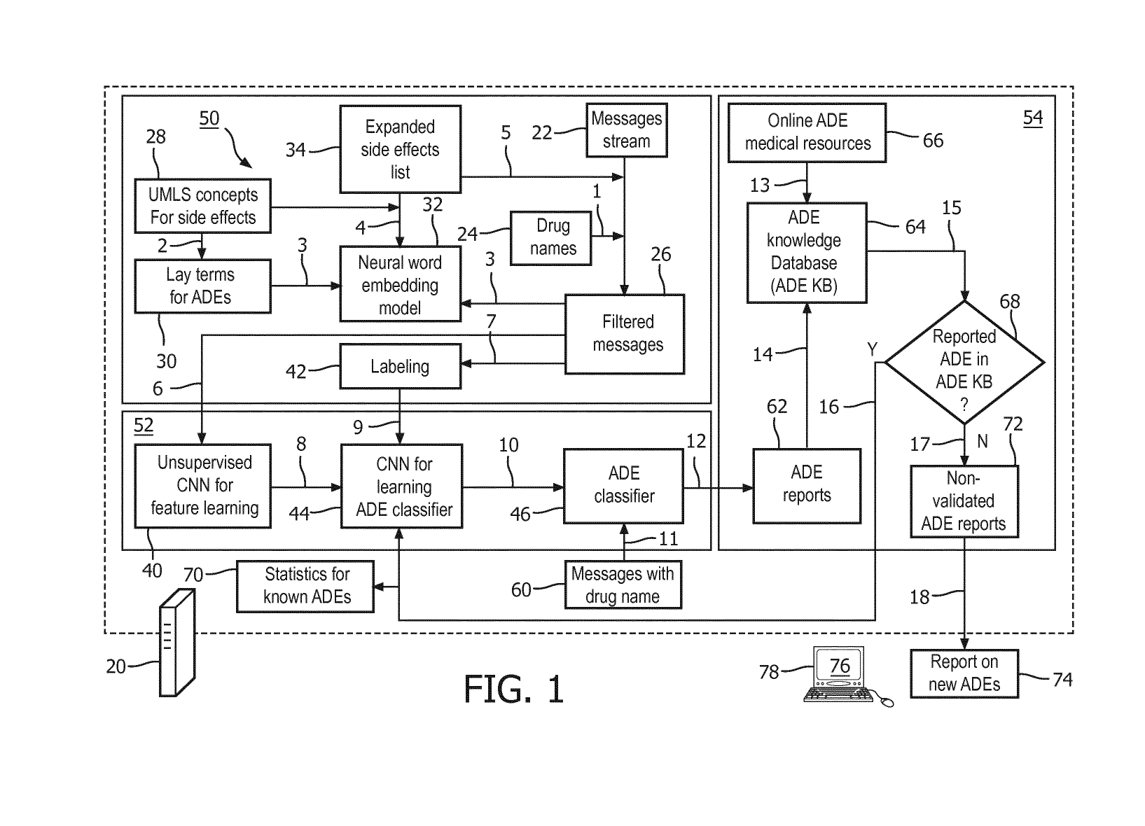

With reference now to FIG. 1, an illustrative pharmacovigilance device providing ADE monitoring and reporting is described. In the example of FIG. 1, locations 1-18 of the diagrammatic representation are described in detail below, with diagrammatically indicated components or other entities also labeled. The ADE monitoring and reporting device is suitably implemented on a computer 20, e.g. a network server computer (“server”), a computing cluster, a cloud computing resource, or so forth. It will be further appreciated that disclosed ADE monitoring and reporting device embodiments may be implemented as a non-transitory storage medium storing instructions readable and executable by such a computer 20 (i.e. instructions that program the computer 20) to perform the disclosed operations. The non-transitory storage medium may, for example, comprise a hard disk drive or other magnetic storage medium, and/or an optical disk or other optical storage medium, and/or a FLASH memory, solid state drive or other electronic storage medium, various combinations thereof, or so forth.

As indicated at 1, publicly available social media messages 22 are collected using streaming and/or restful application program interfaces (APIs) in real time. The messages are filtered using a list of drug names 24, e.g. derived from UMLS. It may be noted that a single drug may have two or more different drug names, e.g. some drugs are named differently in different countries, and/or there may be a generic drug name or the drug may sometimes be referred to by its active ingredient or active agent; the list of drug names 24 preferably captures such regional and/or generic drug names. Since drug names are often long and complex, the list of drug names 24 may also include some common misspellings and/or shortened versions of drug names. This is beneficial since social media messages are sometimes not carefully proofread prior to posting so that occasional drug name misspellings can be expected; similarly, social media posts sometimes use shorthand names, especially in social media such as Twitter that limit the number of words and/or characters per message. The output is a set of filtered messages 26 that contain drug names and/or mention at least one ADE (identified as described next starting with 2). Note that since the filtered messages 26 form a database for training an ADE detector, the list of drug names 24 is not limited to the particular drug whose ADEs are being monitored by the ADE monitoring and reporting device of FIG. 1.

As indicated at 2, a side effects terminology database is created using a medical terminology reference 28 such as the UMLS Metathesaurus and/or one or more other well-curated medical and pharmacological dictionaries. The side effects terminology database is preferably expanded by replacing or augmenting medical terminologies in side effect phrases with the corresponding lay terms or phrases 30 curated from a collection of available online medical-lay mapping dictionaries or other sources. For example, a lay term for “hallucination” is “seeing things”, and thus the phrase “seeing things” can be added to the side effects list. Augmentation by lay terms advantageously improves the ability to detect health conditions described in non-technical and conversational language of the type typically presented in social media posts. As indicated at 3, a neural embedding algorithm 32 receives as input the filtered messages 26 and the expanded side effects list (from 2) as training data for a model, builds a vocabulary, and learns vector representations of words based on the context (semantic and syntactic relationships) of words present in sentences.

Given a word, the model predicts nearby words. This unsupervised training 32 does not require labeled data and therefore can be efficiently trained on large data sets. As indicated at 4, the neural word embedding model 32 is used to search for similar phrases for each side effect. The similar phrases are appended to the original side effects list to further enrich the corpus side effects terminology with phrases describing ADEs in non-technical terms so as to build up an expanded corpus of ADE terminology 34. As indicated at 5, the expanded side effects 34 is used to filter messages of the message stream 22 to identify messages that mention at least one ADE.

As indicated at 6, the filtered messages 26 are used as input to an unsupervised feature learning module 40 which in the illustrative example employs a Convolutional Neural Network (CNN) architecture. A sub-set or all of the filtered messages 26 are further labelled in a manual labeling operation 42 by expert annotators (e.g. pharmacologists, clinicians, or other medical professionals) based on a binary classification (“ADE” or “non-ADE”). The “ADE” label indicates that the message contains a mention of a drug name and also mentions a side effect (with negative polarity) experienced while on a medication. A “non-ADE” label indicates the message indicates the absence of any mentions of either a drug name or any ADE.

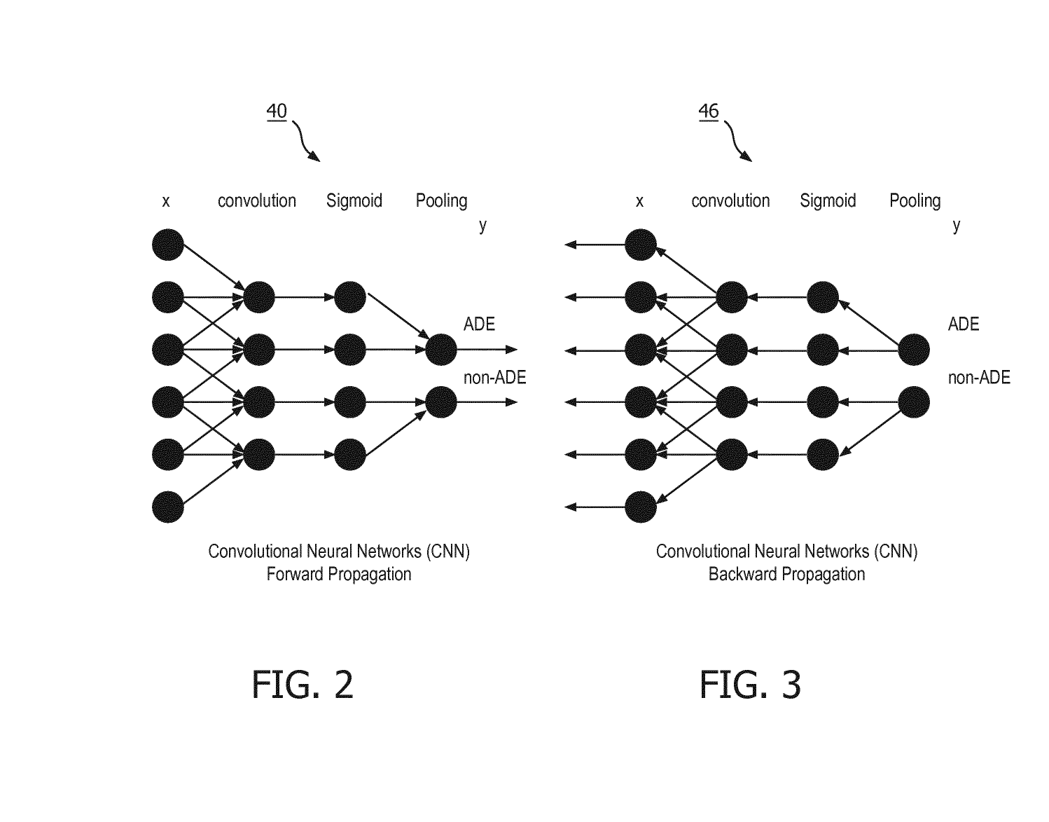

With continuing reference to FIG. 1 and with further reference to FIG. 2, as indicated at 8, in the unsupervised feature learning module 40, a CNN is trained to learn embeddings of phrases (n-grams) from the unlabeled text data. Training data are first generated by converting ADE-descriptive phrases such as “can't sleep” or “loss of appetite” to low-dimensional bag-of-word or bag-of-n-gram feature vectors and then, for a given phrase, training to predict context (adjacent phrases). The learned vector representations of phrases are used as features to identify ADEs in a supervised CNN classifier 44 in the next step. As shown in FIG. 2, the feed-forward neural network 40 (i.e. the CNN 40 for feature extraction) receives an n-gram x at far left, which is to be classified as either “ADE” or “non-ADE”. The CNN 40 includes a convolutional layer followed by non-linearity (e.g., a sigmoid, ReLU, tanh, or other non-linear function), followed by a pooling layer (e.g. a max or average pooling layer) which outputs a binary label y having either the value “ADE” or the value “non-ADE”.

With continuing reference to FIG. 1 and with further reference to FIG. 3, as indicated at 9 and 10, in a second phase of semi-supervised CNN architecture, the supervised CNN 44 is trained with embeddings of phrases (learned from unsupervised training as indicated at 8) and annotated ADE data (messages and their labels provided by the manual labeling 42) to produce an ADE classifier 46. As shown in FIG. 3, the network parameters for the supervised CNN 44 are learned by back-propagating classification errors (labels y which are incorrect) through the subsampling and convolution layers, and adjusting network weights to reduce the overall cost.

The portions of the ADE monitoring and reporting device of FIG. 1 described thus far can be approximately divided into a data collection and preparation portion 50 that generates the training data, and a deep learning component 52 that learns the semi-supervised ADE classifier 46. The approach leverages a large dataset of social media messages, most of which can be unlabeled and used for training the first phase ADE classification 40. Advantageously, only a small sub-set of this data set needs to be labeled by the manual labeling 42 in order to provide the feedback for adjusting the network weights in the supervised training phase 44.

The illustrative embodiment employs CNN as the ADE classifier; however, other types of classifiers are alternatively contemplated, such as Support Vector Machine (SVM) classifiers, kernel classifiers, or so forth. Such alternative classifiers may be trained using semi-supervised training (as in the illustrative embodiment) or using fully supervised training. In one such alternative approach, a binary SVM classifier is trained to detect each different ADE in the expanded list 34 (with the binary SVM outputting “1” for “ADE” and “0” for “non-ADE”) and the overall ADE classifier is then constructed using a logical “OR” of the outputs of these binary SVM classifiers.

After the data collection/preparation and training phases 50, 52, the resulting ADE classifier 46 is used in an inference phase to detect ADEs in messages containing the name of the drug undergoing ADE monitoring. This portion of the ADE monitoring and reporting device of FIG. 1 employs a knowledge-based validation component 54 which is described next.

As indicated at 11 and 12, a message 60 containing the name of the monitored drug (also referred to herein as a “drug-related message”) is classified by the ADE classifier 46. More particularly, a received social media message 60 is first processed to determine whether it contains a mention of the drug being monitored by the ADE monitoring and reporting device. Since a given drug is usually identified by one or, at most, a few different names (different regional names, and/or an active ingredient name, and/or a generic drug name), the identification of a message that contains at least one mention of the monitored drug entails searching for whether the message contains any of these few drug names (and possibly one or more common misspellings and/or one or more common shorthand or shortened versions of the drug name such as may be expected to occur in relatively informal social media postings). Those messages that contain at least one mention of the monitored drug are inputs to the ADE classifier 46, which classifies each message as ADE or non-ADE and identifies n-grams (ADE phrases) within the message that is indicative of the classification. Each such ADE identification in a message 60 containing the drug name constitutes an ADE report 62.

As indicated at 13, an ADE knowledge database 64 is created by combining drug-side effect data from one or more online medical knowledge resources 66, such as regulatory authorities, drug and side effect data from public access medical websites such as WebMD, user-reported data on FDA Adverse Event Reporting System such as FAERS, PubMed articles, or so forth. As indicated at 14, the ADE reports 62 are validated against evidence in the ADE knowledge database 64. This validation may entail, for example, generating the ADE knowledge database 64 as a set of known ADEs for the monitored drug from information in the medical resources 66, and validating an ADE report 62 if it is one of these known ADEs. More generally, correlation of ADE can be measured by matching the monitored drug name and measuring semantic similarities of negative side effect phrases found in the social media message 60 containing the ADE report 62 against the ADEs of the set of known ADEs defined in the ADE knowledge base 64 for the monitored drug. In embodiments in which the drug-related message 60 is decomposed into n-grams that are classified by the ADE classifier 46, this entails identifying the ADE n-grams (i.e. the n-grams that are classified as ADEs) in the set of known ADEs for the monitored drug which are stored in the ADE knowledge base 64.

As indicated at 15 and 16, when the ADE report 62 from a social media message semantically correlates with evidence found in the ADE knowledge base 64, the ADE report is validated at decision 68 and this validated ADE report is optionally sent back to the supervised classifier training block 44 in a feedback loop to fine tune the model parameters so as to make the ADE classifier 46 more robust. Additionally, or alternatively, statistics 70 for the validated ADE reports in social media for the monitored drugs can be collected to provide information on relative occurrence frequencies of known ADEs in the ADE reports that pass the validating. For example, ADE reports that pass the validating may be grouped by known ADE, and the frequency of each ADE is the number of messages reporting the known ADE (or, alternatively, the number of unique patients reporting the known ADE). These counts can be normalized to provide relative frequencies.

As indicated at 17, when an ADE report 62 does not match evidence in the ADE knowledge base 64 (that is, the ADE is not a known side effect of the monitored drug) then the non-validated ADE report is stored in a repository 72 of non-validated ADE reports. As indicated at 18, if this non-validated ADE is reported in multiple social media messages and if the number of such ADE reports exceeds an empirical threshold δ, then this ADE is identified as a previously unknown ADE. The threshold δ is typically for the total number of social media messages mentioning the ADE along with the monitored drug. In an alternative embodiment, the threshold δ is for the total number of unique patients receiving the monitored drug that report the ADE in social media. This latter approach advantageously can filter out patients who are very active in social media and hence may mention the ADE in connection with the monitored drug in many different social media posts; however, thresholding on unique patients entails identification of the patient receiving the monitored drug in the social media message. One approach is to identify the patient receiving the monitored drug as the user name of the user who posted the social media message. This approach is inexact because individuals sometimes use different user names on different social media sites, and also because the poster may be describing the ADE in some other person. The latter source of error in patient identification can be reduced by deep semantic analysis of the natural language text of the message, albeit at the cost of increased computational complexity. As an example, if threshold δ=10 and if at least 10 different messages (or, in the alternative embodiment, 10 different, i.e. unique, patients) report the same ADE that is not found in the knowledge base 64, then this ADE is designated as a previously unrecognized ADE of the monitored drug and hence is included in a report 74 on new (i.e. previously unrecognized) ADEs of the monitored drug. Optionally, the knowledge base 64 is periodically updated and if a previously unrecognized ADE now appears in the updated knowledge base 64 it is then removed from the report 74. The report 74 advantageously provides improved pharmacovigilance by providing rapid identification of previously unrecognized ADEs.

The report 74 may be variously used. It may, for example, be printed or stored as a PDF file and viewed on a display 76 of a computer or computer terminal 78, or its contents may be cut/pasted into a post-market FDA report being prepared by an employee of the pharmaceutical company. In some embodiments, the report 74 also summarizes the information statistics 70 on relative occurrence frequencies of known ADEs, so as to provide information on the (relative) prevalence of these known ADEs in the actual post-market patient population.

The ADE monitoring and reporting device of FIG. 1 can be employed to monitor ADE reports on social media for various drugs, merely by inputting social media messages 60 mentioning the various drugs to be monitored, and sorting the results 70, 72 by the mentioned drug. It should also be noted that “drug” may optionally encompass a family or class of drugs, for example the ADE monitoring and reporting device could be used to monitor ADEs of a class of steroid-based drugs, or more generally a class of drugs that all employ the same active ingredient.

It should also be noted that since the preparatory and training components 50, 52 employ the listing of drug names 24 and ADE terminology 28, 30 which are not specific to the particular monitored drug, the resulting ADE classifier 46 may be used (or re-used) for ADE monitoring/reporting for various different specific monitored drugs.

In the device of FIG. 1, validated ADE reports are fed back to the CNN learner 44 for use in tuning as indicated at 16. By contrast, non-validated ADE reports are not fed back to the CNN learner 44 for tuning. This is because it is not known whether or not the non-validated ADE report is correct. On the other hand, the non-validated ADE report is useful if it is confirmed by way of contributing to an aggregation of non-validated ADE reports indicating the same ADE, as this is evidence that the non-validated ADE report is reporting on a previously unrecognized ADE of the monitored drug.

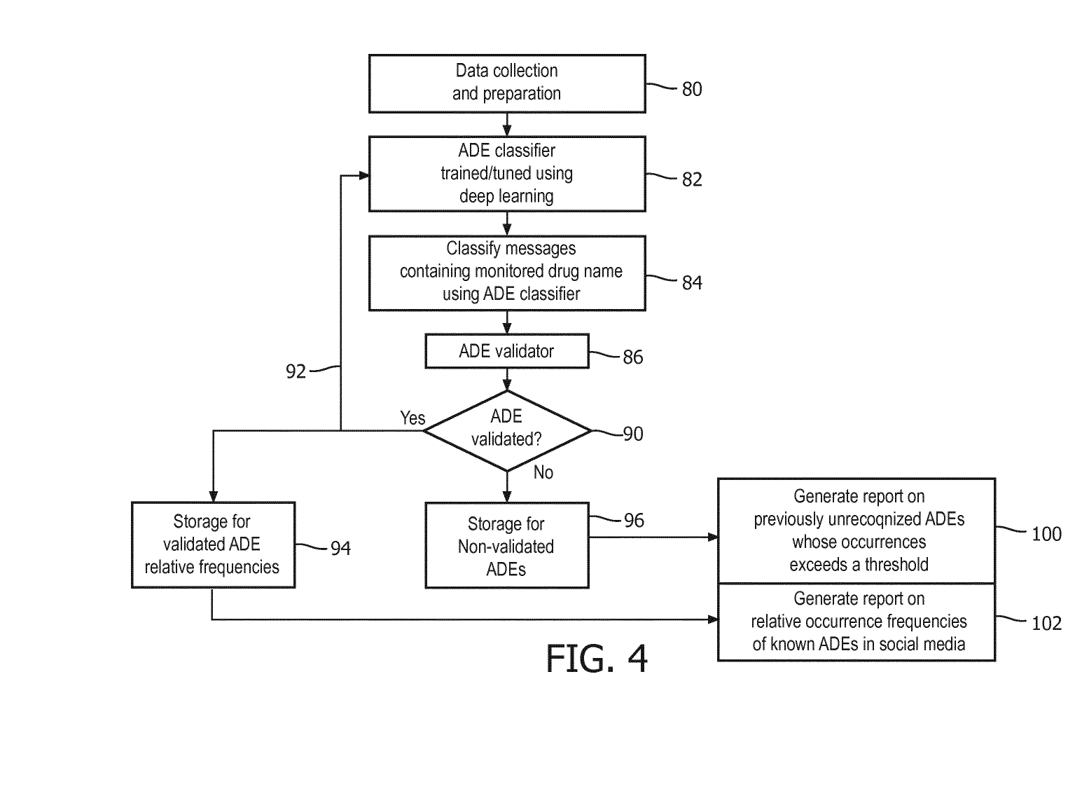

With reference to FIG. 4, a drug monitoring and reporting method suitably performed by the device of FIG. 1 is described. In an operation 80, the social media messages collection and processing is performed by the device portion 50 to generate training data (filtered messages 26 with selected annotation by the labeling 42). In an operation 82, the ADE classifier 46 is trained using the deep learning component 52. In an operation 84, social media messages containing the monitored drug name (or, containing one or more of the regional, shorthand, or other variants of the drug name) are identified and classified as to whether they contain at least one ADE report 62 using the ADE classifier 46. In an operation 86, each ADE report 62 is validated using the validation portion 54 of the device. At a decision 90, if the ADE report 62 is validated then this validated result is fed back 92 to update the classifier training 82, and/or the ADE report for the known ADE is added to storage 94 of the validated (i.e. known) ADE relative frequencies. On the other hand, if at the decision 90 the ADE report 62 is non-validated then the non-validated ADE report is added to the storage 96 of non-validated ADE reports. In an operation 100, a report is generated on previously unrecognized ADEs identified via the social media monitoring. The previously unrecognized ADEs are those whose ADE reports in the social media exceed some threshold δ on the number of social media messages mentioning both the monitored drug and the ADE. In an alternative embodiment the previously unrecognized ADEs are those for which ADE reports indicate some threshold 6 of unique patients are reporting the ADE in conjunction with the monitored drug on social media. In an operation 102, a report is optionally generated on relative reporting frequencies (i.e. occurrence frequencies) of known ADEs in the ADE reports that pass the validating.

In some embodiments, it is contemplated to omit the validation portion 54 of the ADE monitoring and reporting device. In such embodiments, all ADE reports are suitably logged, and a report may be made on the detected ADEs and their relative frequencies of occurrence in social media messages.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. An adverse drug event (ADE) monitoring and reporting device comprising:
a computer programmed to perform an ADE monitoring and reporting method including:
detecting drug-related messages in one or more social media message streams as messages that include a name of a monitored drug;
extracting ADE reports from the drug-related messages using an ADE classifier, wherein the ADE classifier comprises a convolutional neural network (CNN) classifier trained on n-grams extracted from messages from the one or more social media message streams to classify the messages as to whether they report an ADE using the n-grams as features;
validating the extracted ADE reports by comparison with known ADEs of the monitored drug stored in an ADE knowledge base;
collecting extracted ADE reports that fail the validating in a non-validated ADE reports database; and
generating a report including information on at least one previously unrecognized ADE for which extracted ADE reports in the non-validated ADE reports database satisfies a previously unrecognized ADE criterion.

2. The ADE monitoring and reporting device of claim 1 wherein the ADE monitoring and reporting method the computer is programmed to perform further includes:
tuning the ADE classifier using extracted ADE reports that pass the validating while not tuning the ADE classifier using extracted ADE reports that fail the validating.

3. The ADE monitoring and reporting device of claim 1 wherein the ADE monitoring and reporting method the computer is programmed to perform further includes:
grouping ADE reports that pass the validating by known ADE;
wherein the generated report further includes information on relative occurrence frequencies of known ADEs in the ADE reports that pass the validating.

4. The ADE monitoring and reporting device of claim 3 wherein the extracted ADE reports include identification of patients receiving the monitored drug and the relative occurrence frequencies of known ADEs are for unique patients receiving the monitored drug.

5. (canceled)

6. The ADE monitoring and reporting device of claim 1 wherein the ADE classifier is trained to detect ADEs represented by ADE terminology including lay terms for ADEs.

7. The ADE monitoring and reporting device of claim 1 wherein:
the extracting includes extracting ADE n-grams representing ADEs from the drug-related messages; and
the validating includes identifying the ADE n-grams in the ADE knowledge base.

8. The ADE monitoring and reporting device of claim 1 wherein the previously unrecognized ADE criterion comprises the number of unique patients having at least one non-validated ADE report indicating the previously unrecognized ADE in the non-validated ADE reports database exceeding a threshold.

9. The ADE monitoring and reporting device of claim 1 wherein the previously unrecognized ADE criterion comprises the number of non-validated ADE reports indicating the previously unrecognized ADE in the non-validated ADE reports database exceeding a threshold.

10. The ADE monitoring and reporting device of claim 1 wherein the detecting includes:
detecting drug-related messages from the one or more social media message streams as messages that include any of a plurality of names of the monitored drug.

11. A non-transitory storage medium storing instructions readable and executable by a computer to perform an adverse drug event (ADE) monitoring and reporting method for a monitored drug having a set of known ADEs, the method comprising:
identifying drug-related messages in one or more social media message streams wherein each drug-related message includes a name of the monitored drug;
extracting ADE reports from the drug-related messages by classification of the drug-related messages using n-grams extracted from the drug-related messages as features of an ADE classifier, wherein the ADE classifier comprises a convolutional neural network (CNN) classifier trained on n-grams extracted from messages from the one or more social media message streams; and
identifying a previously unrecognized ADE that is not in the set of known ADEs for the monitored drug in response to an accumulation of extracted ADE reports indicating the previously unrecognized ADE.

12. The non-transitory storage medium of claim 11 wherein:
the extracting includes extracting patients who are subjects of the ADE reports; and
the identifying comprises identifying the previously unrecognized ADE in response to an accumulation of extracted ADE reports indicating the previously unrecognized ADE for at least a threshold number of different patients.

13. The non-transitory storage medium of claim 11 wherein the identifying comprises identifying the previously unrecognized ADE in response to the number of extracted ADE reports indicating the previously unrecognized ADE exceeding a threshold.

14. (canceled)

15. (canceled)

16. (canceled)

17. (canceled)

18. An adverse drug event (ADE) monitoring and reporting method performed for a monitored drug, the method comprising:
identifying drug-related messages that include a name of the monitored drug;
extracting ADE reports from the identified ADE reporting messages by classifying text of the drug-related messages using an ADE classifier, wherein the ADE classifier comprises a convolutional neural network (CNN) classifier trained on n-grams extracted from messages from the one or more social media message streams to classify the messages as to whether they report an ADE using the n-grams as features; and
outputting a report on the extracted ADE reports.

19. (canceled)

20. (canceled)

21. (canceled)
